(19) United States
(12) Patent Application Publication
(10) Pub. No.: US 2022/0128577 A1
(43) Pub. Date: Apr. 28, 2022

(54) Methods for Diagnosing Alzheimer's Disease Based on Cell Growth Rate, Size and Protein Amount

(71) Applicant: NeuroDiagnostics LLC, Rockville, MD (US)

(72) Inventors: Florin Valentin Chirila, Morgantown, WV (US); Daniel L. Alkon, Chevy Chase, MD (US)

(73) Assignee: NeuroDiagnostics LLC, Rockville, MD (US)

(21) Appl. No.: 17/429,374

(22) PCT Filed: Feb. 26, 2020
(86) PCT No.: PCT/US2020/019812

§ 371 Date: Aug. 9, 2021

Related U.S. Application Data
(60) Provisional application No. 62/810,659, filed on Feb. 26, 2019.

(51) Int. Cl. G01N 33/68; C12N 5/077; G01N 33/50; C12N 5/0781

(52) U.S. Cl. CPC G01N 33/6896; C12N 5/0656; G01N 33/5044; C12N 5/0635; G01N 2800/2821; G01N 33/5091; G01N 2800/50; G01N 2570/00; G01N 33/5052

(57) ABSTRACT

This invention provides methods for diagnosing Alzheimer's disease in a symptomatic human subject. These methods comprise measuring the growth rate, size and/or protein amount of a subject's skin fibroblasts and/or lymphocytes, and determining whether these values differ in certain ways from those of corresponding non-Alzheimer's disease dementia cells. This invention also provides methods for determining whether an asymptomatic human subject is at risk from becoming afflicted with Alzheimer's disease.

This application claims the benefit of U.S. Provisional Application No. 62/810,659, filed Feb. 26, 2019, the contents of which are incorporated herein by reference.

Throughout this application, various publications are cited. The disclosure of these publications is hereby incorporated by reference into this application to describe more fully the state of the art to which this invention pertains.

### BACKGROUND OF THE INVENTION

Alzheimer's disease (“AD”) has long been the subject of considerable efforts to develop accurate diagnostic methods, as well as therapeutic methods. Despite these efforts, there is an unmet need for methods of accurately diagnosing AD and differentiating it from non-Alzheimer's dementia (“non-ADD”).

### SUMMARY OF THE INVENTION

This invention provides a method for diagnosing Alzheimer's disease in a symptomatic human subject comprising (a) culturing skin fibroblasts from the subject under skin fibroblast growth-permitting conditions and (b) measuring the growth rate of the subject's skin fibroblasts, whereby the subject is afflicted with Alzheimer's disease if the growth rate of the subject's skin fibroblasts is less, by at least one standard deviation, than the growth rate of non-Alzheimer's disease dementia skin fibroblasts cultured under the same skin fibroblast growth-permitting conditions.

This invention also provides a method for diagnosing Alzheimer's disease in a symptomatic human subject comprising (a) culturing lymphocytes from the subject under lymphocyte growth-permitting conditions and (b) measuring the growth rate of the subject's lymphocytes, whereby the subject is afflicted with Alzheimer's disease if the growth rate of the subject's lymphocytes is less, by at least one standard deviation, than the growth rate of non-Alzheimer's disease dementia lymphocytes under the same lymphocyte growth-permitting conditions.

This invention further provides a method for diagnosing Alzheimer's disease in a symptomatic human subject comprising measuring the size of the subject's skin fibroblasts under skin fibroblast size-measuring conditions, whereby the subject is afflicted with Alzheimer's disease if the size of the subject's skin fibroblasts is greater, by at least one standard deviation, than the size of non-Alzheimer's disease dementia skin fibroblasts measured under the same skin fibroblast size-measuring conditions.

This invention still further provides a method for diagnosing Alzheimer's disease in a symptomatic human subject comprising measuring the size of the subject's lymphocytes under lymphocyte size-measuring conditions, whereby the subject is afflicted with Alzheimer's disease if the size of the subject's lymphocytes is greater, by at least one standard deviation, than the size of non-Alzheimer's disease dementia lymphocytes measured under the same lymphocyte size-measuring conditions.

This invention provides a method for diagnosing Alzheimer's disease in a symptomatic human subject comprising (a) (i) culturing skin fibroblasts from the subject under skin fibroblast growth-permitting conditions and (ii) measuring the growth rate of the subject's skin fibroblasts; and (b) measuring the size of the subject's skin fibroblasts under skin fibroblast size-measuring conditions, whereby the subject is afflicted with Alzheimer's disease if
the growth rate of the subject's skin fibroblasts is less, by at least one standard deviation, than the growth rate of non-Alzheimer's disease dementia skin fibroblasts cultured under the same skin fibroblast growth-permitting conditions, and/or
the size of the subject's skin fibroblasts is greater, by at least one standard deviation, than the size of non-Alzheimer's disease dementia skin fibroblasts measured under the same skin fibroblast size-measuring conditions.

This invention also provides a method for diagnosing Alzheimer's disease in a symptomatic human subject comprising (a) (i) culturing lymphocytes from the subject under lymphocyte growth-permitting conditions and (ii) measuring the growth rate of the subject's lymphocytes; and (b) measuring the size of the subject's lymphocytes under lymphocyte size-measuring conditions, whereby the subject is afflicted with Alzheimer's disease if
the growth rate of the subject's lymphocytes is less, by at least one standard deviation, than the growth rate of non-Alzheimer's disease dementia lymphocytes cultured under the same lymphocyte growth-permitting conditions, and/or
the size of the subject's lymphocytes is greater, by at least one standard deviation, than the size of non-Alzheimer's disease dementia lymphocytes measured under the same lymphocyte size-measuring conditions.

This invention further provides a method for diagnosing Alzheimer's disease in a symptomatic human subject comprising measuring the protein amount of the subject's skin fibroblasts under protein amount-measuring conditions, whereby the subject is afflicted with Alzheimer's disease if the protein amount of the subject's skin fibroblasts is lower, by at least one standard deviation, than the average protein amount of non-Alzheimer's disease dementia skin fibroblasts measured under the same protein amount-measuring conditions.

This invention still further provides a method for diagnosing Alzheimer's disease in a symptomatic human subject comprising measuring the protein amount of the subject's lymphocytes under protein amount-measuring conditions, whereby the subject is afflicted with Alzheimer's disease if the protein amount of the subject's lymphocytes is lower, by at least one standard deviation, than the average protein amount of non-Alzheimer's disease dementia lymphocytes measured under the same protein amount-measuring conditions.

This invention provides a method for determining whether an asymptomatic human subject is at risk from becoming afflicted with Alzheimer's disease comprising (a) culturing skin fibroblasts from the subject under skin fibroblast growth-permitting conditions and (b) measuring the growth rate of the subject's skin fibroblasts, whereby the subject is at risk from becoming afflicted with Alzheimer's disease if the growth rate of the subject's skin fibroblasts is greater than, yet within two standard deviations of, the growth rate of Alzheimer's disease skin fibroblasts cultured under the same skin fibroblast growth-permitting conditions.

This invention also provides a method for determining whether an asymptomatic human subject is at risk from becoming afflicted with Alzheimer's disease comprising (a) culturing lymphocytes from the subject under lymphocyte growth-permitting conditions and (b) measuring the growth rate of the subject's lymphocytes, whereby the subject is at risk from becoming afflicted with Alzheimer's disease if the growth rate of the subject's lymphocytes is greater than, yet within two standard deviations of, the growth rate of Alzheimer's disease lymphocytes under the same lymphocyte growth-permitting conditions.

This invention further provides a method for determining whether an asymptomatic human subject is at risk from becoming afflicted with Alzheimer's disease comprising measuring the size of the subject's skin fibroblasts under skin fibroblast size-measuring conditions, whereby the subject is at risk from becoming afflicted with Alzheimer's disease if the size of the subject's skin fibroblasts is lower than, yet within two standard deviations of, the size of Alzheimer's disease skin fibroblasts measured under the same skin fibroblast size-measuring conditions.

This invention further provides a method for determining whether an asymptomatic human subject is at risk from becoming afflicted with Alzheimer's disease comprising measuring the size of the subject's lymphocytes under lymphocyte size-measuring conditions, whereby the subject is at risk from becoming afflicted with Alzheimer's disease if the size of the subject's lymphocytes is lower than, yet within two standard deviations of, the size of Alzheimer's disease lymphocytes measured under the same lymphocyte size-measuring conditions.

This invention further provides a method for determining whether an asymptomatic human subject is at risk from becoming afflicted with Alzheimer's disease comprising (a) (i) culturing skin fibroblasts from the subject under skin fibroblast growth-permitting conditions and (ii) measuring the growth rate of the subject's skin fibroblasts; and (b) measuring the size of the subject's skin fibroblasts under skin fibroblast size-measuring conditions, whereby the subject is at risk from becoming afflicted with Alzheimer's disease if
the growth rate of the subject's skin fibroblasts is greater than, yet within two standard deviations of, the growth rate of Alzheimer's disease skin fibroblasts cultured under the same skin fibroblast growth-permitting conditions, and/or
the size of the subject's skin fibroblasts is lower than, yet within two standard deviations of, the size of Alzheimer's disease skin fibroblasts measured under the same skin fibroblast size-measuring conditions.

This invention further provides a method for determining whether an asymptomatic human subject is at risk from becoming afflicted with Alzheimer's disease comprising (a) (i) culturing lymphocytes from the subject under lymphocyte growth-permitting conditions and (ii) measuring the growth rate of the subject's lymphocytes; and (b) measuring the size of the subject's lymphocytes under lymphocyte size-measuring conditions, whereby the subject is at risk from becoming afflicted with Alzheimer's disease if
the growth rate of the subject's lymphocytes is greater than, yet within two standard deviations of, the growth rate of Alzheimer's disease lymphocytes cultured under the same lymphocyte growth-permitting conditions, and/or
the size of the subject's lymphocytes is lower than, yet within two standard deviations of, the size of Alzheimer's disease lymphocytes measured under the same lymphocyte size-measuring conditions.

This invention still further provides a method for determining whether an asymptomatic human subject is at risk from becoming afflicted with Alzheimer's disease comprising measuring the protein amount of the subject's skin fibroblasts under protein amount-measuring conditions, whereby the subject is at risk from becoming afflicted with Alzheimer's disease if the protein amount of the subject's skin fibroblasts is greater than, yet within two standard deviations of, the average protein amount of Alzheimer's disease skin fibroblasts measured under the same protein amount-measuring conditions.

Finally, this invention provides a method for determining whether an asymptomatic human subject is at risk from becoming afflicted with Alzheimer's disease comprising measuring the protein amount of the subject's lymphocytes under protein amount-measuring conditions, whereby the subject is at risk from becoming afflicted with Alzheimer's disease if the protein amount of the subject's lymphocytes is greater than, yet within two standard deviations of, the average protein amount of Alzheimer's disease lymphocytes measured under the same protein amount-measuring conditions.

### BRIEF DESCRIPTION OF THE FIGURES

FIGS. 1A-1D

These Figures show impaired growth rate and large cell size in Alzheimer's disease patients (n=17) when compared with Non-ADD patients (n=4). (1A) Growth rate versus cell size for one AD subject, one Non-ADD subject and four Fetal Bovine Serum (“FBS”) lots. Regardless of the FBS lot, the separation is statistically significant for both measures, growth rate (P<0.004 shown as a horizontal line) and cell size (P<0.004 not shown); T-test two tailed equal variance. The line represents the cut-off for the growth rate and it is at 5,600. (1B) Growth rate versus cell size for 21 subjects with autopsy confirmation, 17 Alzheimer's disease patients (squares), 4 Non-ADD patients (circles), and 8 samples from healthy controls (HC) (triangles). The separation is statistically significant for growth rate (P<0.035 shown as a horizontal line) as well as for the cell size (P<0.026 not shown); T-test two tailed unequal variance). These data show (i) an overlap of the growth rate signal for HC and Non-ADD, (ii) a range of signals for the HC from 6,408 to 11,824, and (iii) a gap between the cut-off, 5,600, and the lowest signal for the HC, 6,408. This range of signals for the growth rate of HC, as well as the gap, indicate that Mildly Cognitively Impaired (MCI) patients would be represented in that gap. This range of HC signals would be expected to show disease progression. (1C) Zoom in near the cut-off of 5,600 to demonstrate that the Non-ADD case (circle and arrow) is well above the cut-off line. (1D) Sensitivity and specificity graphs for the growth rate and cell size. A third approach would be to test the uncertain patients like the two in this study (arrows) at a subsequent time of six months to a year, and to check if the diagnosis is unambiguous.

FIGS. 2A and 2B

These Figures show a linear correlation between skin fibroblast size (measured on Matrigel at 30 minutes after plating when cell confluence is less than 10%) and skin fibroblast size (measured in T25 flasks at 85% confluence). The AD cells (squares) are larger than the cells from Non-ADD patients (circles). The cut-off, represented by a vertical line, is 1,900 mm² and was determined as middle of the gap between AD and Non-ADD populations. FIG. 2B represents normalized values for the same measure presented in FIG. 2A.

FIGS. 3A and 3B

These Figures show reduced numbers of B-lymphocytes in AD patients. Specifically, the total number of cells (3A) and the number of viable (i.e., live) cells (3B) are reduced in AD patients (triangles) when compared with Non-Demented Controls (“NDC”) (circles) and Non-ADD patients (squares).

FIGS. 4A-4C

These Figures show reduced growth rate of B-lymphocytes in AD patients. (4A) The growth rate of the AD sample (0.58) is significantly smaller than for the Non-ADD sample (2.84), and the NDC sample (3.55). A cut-off of 1.71 (line) is proposed for this small number of samples for separating AD from Non-ADD and NDC. (4B) The normalized growth rates for the Non-ADD and NDC samples are about 5-fold and 6-fold higher, respectively, than for the AD sample. (4C) This graph shows that the AD samples grow approximately five times slower than the Non-ADD samples and seven times slower than NDC samples.

FIG. 5

This Figure shows impaired growth rate and large cell size in an AD sample (square) compared with a Non-ADD sample (elide) and an NDC sample (triangle).

FIGS. 6A and 6B

These Figures show a reduced protein amount in skin fibroblasts from AD patient samples (squares; n=17) compared with Non-ADD samples (circles; n=4). “Prot. Conc. (+)/Days in C.” is the protein concentration (in ng/mL) for ASPD-stimulated cells (+), divided by the number of days in culture. This is the total protein amount for ASPD-stimulated cells. FIG. 6B shows the normalized values of those shown in FIG. 6A.

FIG. 7

This Figure shows the relationship of Gaussian distribution to standard deviation.

FIG. 8

This Figure shows the relationship between the Gaussian distributions for AD patients and non-ADD patients, and the notions of buffer zone and cutoff.

### DETAILED DESCRIPTION OF THE INVENTION

### Definitions

In this application, certain terms are used which shall have the meanings set forth as follows.

As used herein, “Alzheimer's disease” means a concurrent affliction with the following three symptoms: (i) dementia; (ii) amyloid plaques; and (iii) neurofibrillary tangles. This definition of Alzheimer's disease is the one provided by the National Institute of Neurological Disorders and Stroke (NINDS) of the National Institutes of Health (NIH), and is known as the “gold standard.” Dementia can be diagnosed during life. Cerebral amyloid plaques and neurofibrillary tangles can, for example, be diagnosed during autopsy. All subjects from whom samples were taken and studied, and for which data are presented herein, are autopsy-confirmed AD and non-ADD patients.

As used herein, an asymptomatic human subject is “at risk” from becoming afflicted with Alzheimer's disease if the subject's likelihood of becoming afflicted is, for example, at least 50%, at least 60%, at least 70%, at least 80%, at least 90%, at least 95%, or at least 99%.

As used herein, a human subject is “asymptomatic” if that subject is not suspected of being afflicted with either AD or non-ADD (e.g., a subject not displaying any symptom consistent with either AD or non-ADD, such as dementia).

As used herein, “culturing” lymphocytes is achieved, for example, by conducting the culturing at a temperature and in a growth factor milieu permissive of cell growth. Preferably, culturing lymphocytes is performed under conditions (e.g., those for proliferation) that preserve lymphocyte viability. In one embodiment, the temperature, salinity and protein milieu permissive of cell growth is 37° C., RPMI 1640 Medium with 10% fetal bovine serum (“FBS”) and 1% penicillin (“PS”). In another embodiment of this invention, the lymphocyte-culturing step is performed for more than three hours, more than six hours (e.g., overnight), or for one day, two days, three days, four days, five days, six days, seven days, eight days, nine days or 10 days. Methods for obtaining lymphocytes from a subject's blood are known, and include, for example, flow cytometry, Ficoll (a hydrophilic polysaccharide that separates layers of blood), and gradient centrifugation. Additionally, in the subject methods, the lymphocytes (e.g., B lymphocytes) can be used in immortalized or primary (i.e., non-immortalized) form. Methods for immortalizing lymphocytes (e.g., B lymphocytes) are known, and include, for example, treating the lymphocytes with Epstein-Barr virus (“EBV”).

As used herein, “culturing” skin fibroblasts is achieved, for example, by conducting the culturing at a temperature and in a growth factor milieu permissive of cell growth. Preferably, culturing skin fibroblasts is performed under conditions that preserve skin fibroblasts viability. In one embodiment, the temperature, humidity and protein milieu permissive of cell growth is 37° C., DMEM Medium with 10% fetal bovine serum (“FBS”) and 1% penicillin (“PS”). In another embodiment of this invention, the skin fibroblast-culturing step is performed for more than three hours, more than six hours (e.g., overnight), or for one day, two days, three days, four days, five days, six days, seven days, eight days, nine days or 10 days. Methods for obtaining skin fibroblasts from a subject's blood are known, and include, for example, skin punch biopsy, and growing cells out of explants.

As used herein, cells “derived” from a subject are cells that arise through culturing and/or other physical manipulation performed on cells directly removed from the subject. For example, cultured skin fibroblasts derived from a subject are those skin fibroblasts that arise through culturing a sample of skin cells (e.g., contained in a punch biopsy) directly removed from the subject.

As used herein, “diagnosing Alzheimer's disease”, with respect to a symptomatic human subject, means determining that there is greater than 50% likelihood that the subject is afflicted with Alzheimer's disease. Preferably, diagnosing Alzheimer's disease means determining that there is greater than a 60%, 70%, 80%, 90%, 95% or 99% likelihood that the subject is afflicted with Alzheimer's disease. As used herein, the phrase “determining whether the subject is afflicted with Alzheimer's disease” is synonymous with the phrase “diagnosing Alzheimer's disease.”

As used herein, “diagnosing non-ADD”, with respect to a symptomatic human subject, means determining that there is greater than 50% likelihood that the subject is afflicted with non-ADD. Preferably, diagnosing non-ADD means determining that there is greater than a 60%, 70%, 80%, 90%, 95% or 99% likelihood that the subject is afflicted with non-ADD. As used herein, the phrase “determining whether the subject is afflicted with non-ADD” is synonymous with the phrase “diagnosing non-ADD.” Envisioned as part of this invention, among other things, is the diagnosis of non-ADD in a symptomatic subject by virtue of a negative diagnosis for AD.

As used herein, a gene is “differentially expressed between corresponding cells (e.g., synchronized cells) derived from AD patients and those derived from non-ADD patients” if, for example, the gene's TPM measure in cells (e.g., synchronized cells) derived from AD patients is different than in the same type of cells derived from non-ADD patients (e.g., ones that are synchronized in the same way). For example, gene X would be differentially expressed between corresponding cells (e.g., synchronized cells) derived from AD patients and those derived from non-ADD patients if its TPM measure in cells (e.g., synchronized cells) derived from AD patients were 10 and its TPM measure were 100 in the same type of cells derived from non-ADD patients (e.g., ones that are synchronized in the same way).

As used herein, “lymphocyte growth-permitting conditions” include, without limitation, those described herein for culturing lymphocytes. Particularly envisioned is the use of a temperature, humidity and protein milieu permissive of cell growth (e.g., 37° C., RPMI 1640 Medium with 10% FBS and 1% PS), where the medium is in a flask or other container such that lymphocytes in the medium divide and become more confluent with time.

As used herein, “lymphocyte size-measuring conditions” include, without limitation, the lymphocyte-culturing conditions discussed herein.

As used herein, “measuring the growth rate” of a subject's lymphocytes can be accomplished via any suitable method for measuring cell growth. Such methods include, without limitation, measuring the change (average or otherwise) in cell number/mm³/day, ideally over a time interval representative of the maximum cellular growth rate. An example of a time interval representative of the maximum cellular growth rate would be an interval (measured in minutes, hours or days) at or near the point (i.e., inflection point) where the growth curve's slope would be steepest in a plot of confluence (y-axis) versus time (x-axis). By way of further example, the average growth rate can be determined by measuring, and then averaging, the growth rate occurring between 20% confluence and 80% confluence, between 25% confluence and 75% confluence, between 30% confluence and 70% confluence, between 35% confluence and 65%, confluence between 40% confluence and 60% confluence, or between 45% confluence and 55% confluence. In this context, “confluence” means the extent to which the lymphocytes fill the volume in which they are grown, with 100% confluence meaning that the lymphocytes fill the volume in which they are grown to the maximum extent possible.

As used herein, “measuring the growth rate” of a subject's skin fibroblasts can be accomplished via any suitable method for measuring cell growth. Such methods include, without limitation, measuring the change (average or otherwise) in cell number/cm²/day, ideally over a time interval representative of the maximum cellular growth rate. An example of a time interval representative of the maximum cellular growth rate would be an interval (measured in minutes, hours or days) at or near the point (i.e., inflection point) where the growth curve's slope would be steepest in a plot of confluence (y-axis) versus time (x-axis). By way of further example, the average growth rate can be determined by measuring, and then averaging, the growth rate occurring between 20% confluence and 80% confluence, between 25% confluence and 75% confluence, between 30% confluence and 70% confluence, between 35% confluence and 65%, confluence between 40% confluence and 60% confluence, or between 45% confluence and 55% confluence. In this context, “confluence” means the extent to which the skin fibroblasts cover the surface on which they are grown, with 100% confluence meaning that the skin fibroblasts cover the entire surface on which they are grown.

As used herein, a subject afflicted with “non-Alzheimer's dementia”, also referred to as non-Alzheimer's disease dementia or non-ADD, means a subject showing dementia such as, for example, that which characterizes Parkinson's disease, Huntington's disease and frontotemporal dementia.

As used herein, “non-Alzheimer's disease dementia lymphocytes” are lymphocytes either taken from a non-Alzheimer's disease dementia-afflicted subject, or derived from the lymphocytes of a non-Alzheimer's disease dementia-afflicted subject.

As used herein, “non-Alzheimer's disease dementia skin fibroblasts” are skin fibroblasts either taken from a non-Alzheimer's disease dementia-afflicted subject, or derived from the skin fibroblasts of a non-Alzheimer's disease dementia-afflicted subject.

As used herein, a “population” of cells includes any number of cells permitting the manipulation and study required to assess cell growth rate, cell size or cell protein amount. In one embodiment, the population of cells includes at least 1,000,000 cells. In another embodiment, the population of cells includes between 100,000 cells and 1,000,000 cells, between 10,000 cells and 100,000 cells, between 1,000 cells and 10,000 cells, between 100 cells and 1,000 cells, between 10 cells and 100 cells, and fewer than 10 cells (e.g., one cell).

As used herein, the “protein amount” (also referred to, depending on the circumstances, as “total protein amount”, “protein concentration”, “total protein concentration”, “protein production” and “total protein production”) of a subject's skin fibroblasts includes, without limitation, (i) the amount (e.g., average amount) of total protein or proxy protein in one of the subject's skin fibroblasts (whether cultured or not, and whether isolated or not), and (ii) the amount of total protein or proxy protein in a sample of the subject's skin fibroblasts at a known sample volume and, if cultured, a known confluence. Similarly, the protein amount of a subject's lymphocytes includes, without limitation, (i) the amount (e.g., average amount) of total protein or proxy protein in one of the subject's lymphocytes (whether cultured or not, and whether isolated or not), and (ii) the amount of total protein or proxy protein in a sample of the subject's lymphocytes at a known sample volume and, if cultured, a known confluence. Protein amount can be expressed, for example, in pg/cell, ng, or ng/ml of cell-containing sample. Methods for measuring protein amount are known and include, for example, (i) measuring in a cell or cell population the amount of a proxy protein (i.e., a protein such as tubulin that represents a fixed percentage of a cell's total protein), and (ii) measuring in a cell or cell population the total amount of protein (e.g., via the bicinchoninic acid assay (BCA assay)).

As used herein, the “protein amount-measuring conditions” include, for example, the cell-culturing conditions, the cell growth-permitting conditions and the cell size-measuring conditions described herein. Also, in the subject diagnostic and prognostic methods based on measuring protein amount, it is envisioned, in a preferred embodiment, that control protein amounts (e.g., protein amounts present in control cells, namely, non-ADD and AC cells and cell populations) are determined using control cells under the same conditions (e.g., the same cultured or uncultured state, the same degree of confluence, and the same cell number) as those for the sample cells.

As used herein, measuring the “size” of a subject's skin fibroblasts can be accomplished by any suitable method for measuring cell size. Particularly envisioned is a method for measuring the size of a skin fibroblast by measuring its area as viewed orthogonally to the horizontal surface (e.g., T25 flask bottom) on which it grows. Cell size can be expressed, for example, in pmt.

As used herein, measuring the “size” of a subject's lymphocytes can be accomplished by any suitable method for measuring cell size. Particularly envisioned is a method for measuring lymphocyte size by measuring lymphocyte volume as expressed, for example, in μm³.

As used herein, “skin fibroblast size-measuring conditions” include, without limitation, the skin fibroblast-culturing conditions discussed herein.

As used herein, “skin fibroblast growth-permitting conditions” include, without limitation, those described herein for culturing skin fibroblasts. Particularly envisioned is the use of a temperature, humidity and protein milieu permissive of cell growth (e.g., 37° C., DMEM Medium with 10% FBS and 1% PS), where the medium is in a T25 flask such that skin fibroblasts in the medium adhere to the flask's level, 25 cm² bottom and become more confluent with time. Also envisioned is the use of Matrigel as the surface on which skin fibroblasts become more confluent with time.

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 50 years or older, 55 years or older, 60 years or older, 65 or older, 70 or older, 75 or older, 80 or older, 85 or older, or 90 or older. The instant methods are envisioned for all subjects, preferably humans (and preferably symptomatic).

As used herein, a “symptomatic” human subject is one who is suspected of being afflicted with AD or non-ADD (e.g., a subject displaying at least one symptom consistent with both AD and non-ADD, such as dementia).

As used herein, “synchronizing” a population of cells means placing at least a majority of cells in that population in the same cell cycle stage (namely, in the G1, S, G2 or M stage, and preferably in the G1, S or G2 stage). In one embodiment, synchronizing a population of cells means placing at least 60%, at least 70%, at least 80%, at least 90%, at least 95% or preferably at least 99% of cells in that population in the same cell cycle stage. In another embodiment, synchronizing a population of cells means placing the cells in that population in the same cell cycle stage that they would be in if cultured to over-confluence and then starved. Cell confluence followed by serum starvation typically arrests the cells in the G0/G1 stage.

### Embodiments of the Invention

This invention provides accurate methods for determining whether a human subject is afflicted with AD or non-ADD when the subject is suspected of being afflicted with AD or non-ADD. This invention also provides accurate methods for determining whether an asymptomatic human subject is at risk from becoming afflicted with AD. The subject methods are based, at least in part, on the surprising discovery that the growth rate and size of a patient's skin fibroblasts and lymphocytes, as well as the protein amount of these cells, individually and jointly permit accurately diagnosing the patient as having either AD or non-ADD.

Specifically, this invention provides a method for diagnosing Alzheimer's disease in a symptomatic human subject comprising (a) culturing skin fibroblasts from the subject under skin fibroblast growth-permitting conditions and (b) measuring the growth rate of the subject's skin fibroblasts, whereby the subject is afflicted with Alzheimer's disease if the growth rate of the subject's skin fibroblasts is less, by at least one standard deviation, than the growth rate of non-Alzheimer's disease dementia skin fibroblasts cultured under the same skin fibroblast growth-permitting conditions.

In a preferred embodiment, the subject is afflicted with Alzheimer's disease if the growth rate of the subject's skin fibroblasts is less, by at least one standard deviation, than the average growth rate of non-Alzheimer's disease dementia skin fibroblasts cultured under the same skin fibroblast growth-permitting conditions. In another preferred embodiment, the growth rate of the subject's skin fibroblasts is less, by at least two standard deviations (and ideally by at least three standard deviations), than the growth rate of non-Alzheimer's disease dementia skin fibroblasts cultured under the same skin fibroblast growth-permitting conditions. In a further embodiment, the growth rate of the subject's skin fibroblasts is less, by at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 standard deviations, than the growth rate of non-Alzheimer's disease dementia skin fibroblasts cultured under the same skin fibroblast growth-permitting conditions.

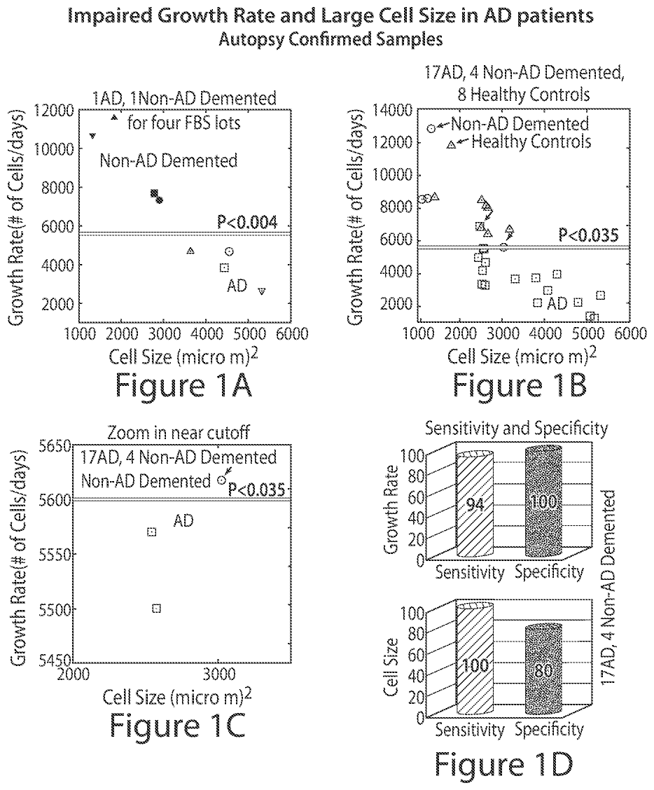

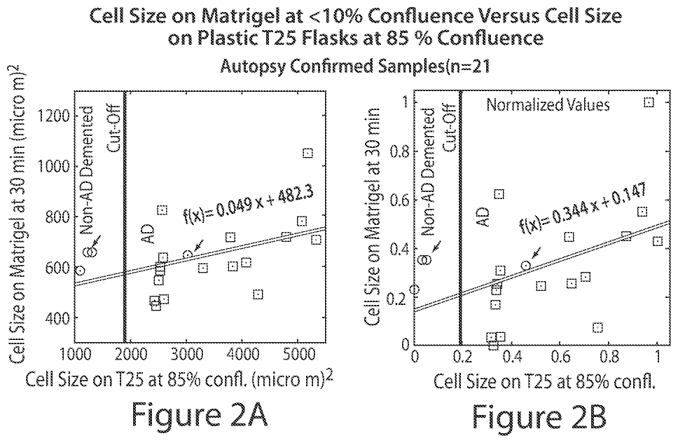

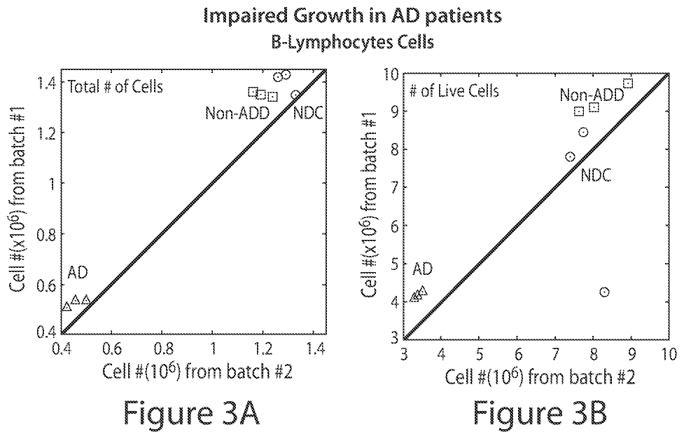

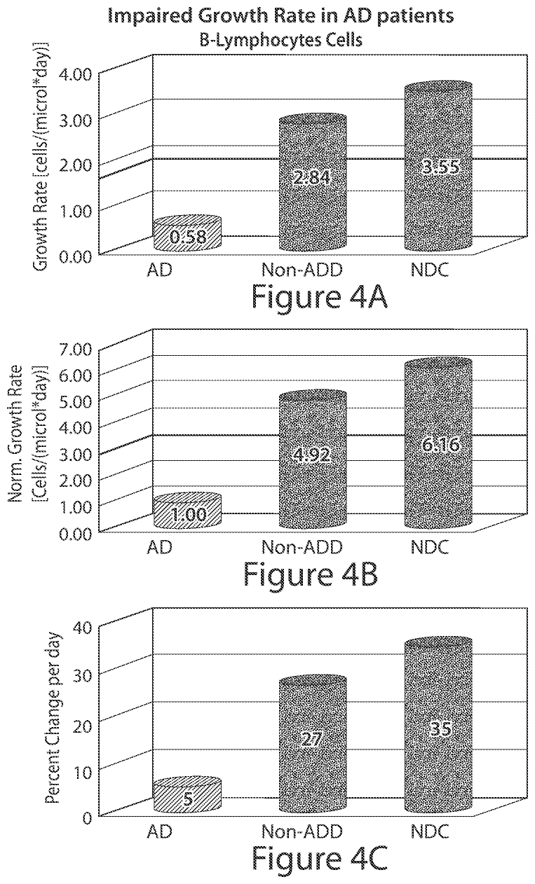

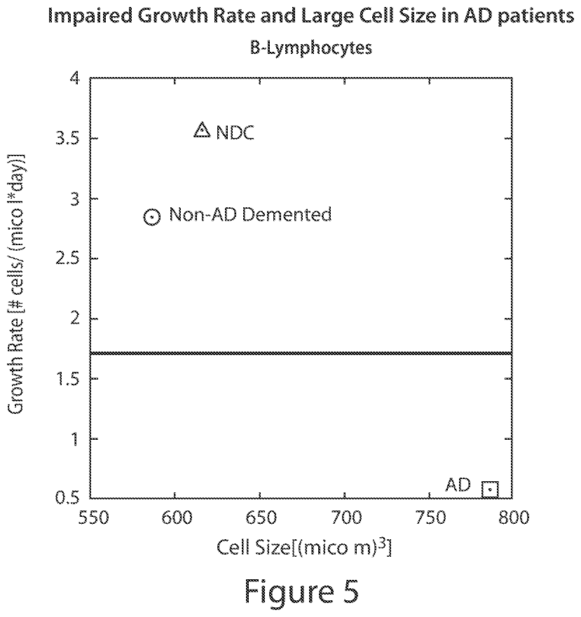

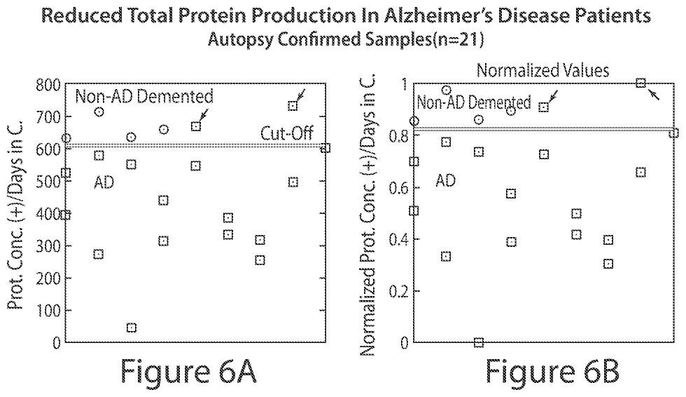

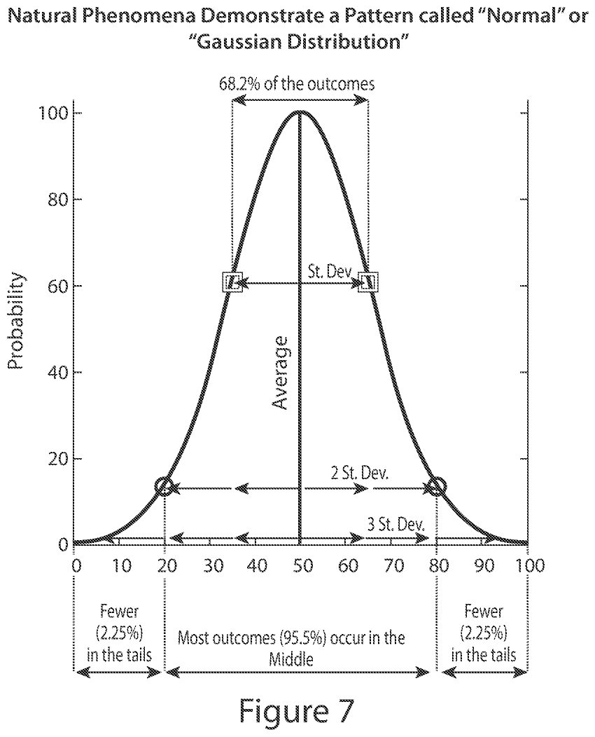

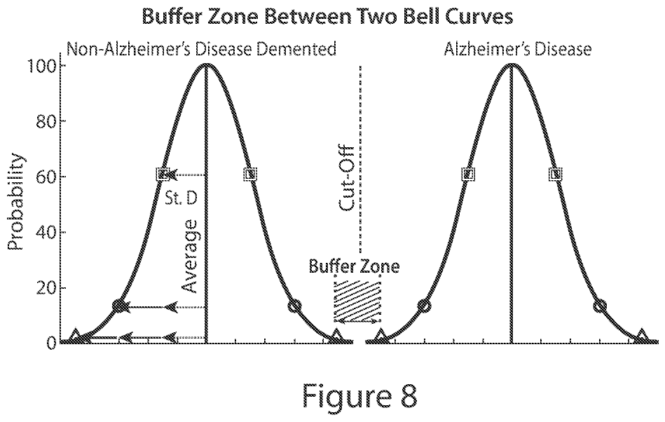

In this method, it is envisioned that under defined skin fibroblast growth-permitting conditions and using defined measuring methods, a growth rate “cutoff” can be determined whereby a skin fibroblast growth rate below that cutoff indicates that the symptomatic subject is afflicted with AD, and a skin fibroblast growth rate above that cutoff indicates that the symptomatic subject is afflicted with non-ADD. An example of this appears in FIGS. 1A-1D, wherein the growth conditions and measuring methods used yield a defined cutoff of 5,600 cells/cm²/day. According to that example, skin fibroblasts grown and measured in the same manner are diagnostic of AD if their growth rate is below the cutoff (e.g., 4,000 cells/cm²/day), while skin fibroblasts grown and measured in the same manner are diagnostic of non-ADD if their growth rate is above the cutoff (e.g., 7,000 cells/cm²/day).

This invention also provides a method for diagnosing Alzheimer's disease in a symptomatic human subject comprising (a) culturing lymphocytes from the subject under lymphocyte growth-permitting conditions and (b) measuring the growth rate of the subject's lymphocytes, whereby the subject is afflicted with Alzheimer's disease if the growth rate of the subject's lymphocytes is less, by at least one standard deviation, than the growth rate of non-Alzheimer's disease dementia lymphocytes under the same lymphocyte growth-permitting conditions. Preferably, the lymphocytes are B-lymphocytes, e.g., immortalized B-lymphocytes.

In a preferred embodiment, the subject is afflicted with Alzheimer's disease if the growth rate of the subject's lymphocytes is less, by at least one standard deviation, than the average growth rate of non-Alzheimer's disease dementia lymphocytes cultured under the same lymphocyte growth-permitting conditions.

In another preferred embodiment, the growth rate of the subject's lymphocytes is less, by at least two standard deviations (and ideally by at least three standard deviations), than the growth rate of non-Alzheimer's disease dementia lymphocytes cultured under the same lymphocyte growth-permitting conditions. In a further embodiment, the growth rate of the subject's lymphocytes is less, by at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 standard deviations, than the growth rate of non-Alzheimer's disease dementia lymphocytes cultured under the same lymphocyte growth-permitting conditions.

In this method, it is envisioned that under defined lymphocyte growth-permitting conditions and using defined measuring methods, a growth rate “cutoff” can be determined whereby a lymphocyte growth rate below that cutoff indicates that the symptomatic subject is afflicted with AD, and a lymphocyte growth rate above that cutoff indicates that the symptomatic subject is afflicted with non-ADD. An example of this appears in FIG. 4A, wherein the growth conditions and measuring methods used yield a defined cutoff of 1.7 cells/μl/day. According to that example, lymphocytes grown and measured in the same manner are diagnostic of AD if their growth rate is below the cutoff (e.g., 1.4 cells/μl/day), while lymphocytes grown and measured in the same manner are diagnostic of non-ADD if their growth rate is above the cutoff (e.g., 2.0 cells/μl/day).

This invention further provides a method for diagnosing Alzheimer's disease in a symptomatic human subject comprising measuring the size of the subject's skin fibroblasts under skin fibroblast size-measuring conditions, whereby the subject is afflicted with Alzheimer's disease if the size of the subject's skin fibroblasts is greater, by at least one standard deviation, than the size of non-Alzheimer's disease dementia skin fibroblasts measured under the same skin fibroblast size-measuring conditions.

In this method, cell size can be measured without first culturing the cells under growth-permitting conditions. Alternatively, cell size can be measured after culturing the cells under growth-permitting conditions.

In a preferred embodiment, the subject is afflicted with Alzheimer's disease if the size of the subject's skin fibroblasts is greater, by at least one standard deviation, than the average size of non-Alzheimer's disease dementia skin fibroblasts measured under the same skin fibroblast size-measuring conditions. In another preferred embodiment, the size of the subject's skin fibroblasts is greater, by at least two standard deviations (and ideally by at least three standard deviations), than the size of non-Alzheimer's disease dementia skin fibroblasts measured under the same skin fibroblast size-measuring conditions. In a further embodiment, the size of the subject's skin fibroblasts is greater, by at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 standard deviations, than the size of non-Alzheimer's disease dementia skin fibroblasts measured under the same skin fibroblast size-measuring conditions.

In this method, it is envisioned that under defined skin fibroblast measuring methods, a size “cutoff” can be determined whereby a skin fibroblast size above that cutoff indicates that the symptomatic subject is afflicted with AD, and a skin fibroblast size below that cutoff indicates that the symptomatic subject is afflicted with non-ADD. An example of this appears in FIGS. 2A and 2B, wherein the measuring methods used yield a defined cutoff of 1,900 μm². According to that example, skin fibroblasts measured in the same manner are diagnostic of AD if their size is above the cutoff (e.g., 2,500 μm²), while skin fibroblasts measured in the same manner are diagnostic of non-ADD if their size is below the cutoff (e.g., 1,500 μm²).

This invention still further provides a method for diagnosing Alzheimer's disease in a symptomatic human subject comprising measuring the size of the subject's lymphocytes under lymphocyte size-measuring conditions, whereby the subject is afflicted with Alzheimer's disease if the size of the subject's lymphocytes is greater, by at least one standard deviation, than the size of non-Alzheimer's disease dementia lymphocytes measured under the same lymphocyte size-measuring conditions. Preferably, the lymphocytes are B-lymphocytes, e.g., immortalized B lymphocytes.

In this method, cell size can be measured without first culturing the cells under growth-permitting conditions. Alternatively, cell size can be measured after culturing the cells under growth-permitting conditions.

In a preferred embodiment, the subject is afflicted with Alzheimer's disease if the size of the subject's lymphocytes is greater, by at least one standard deviation, than the average size of non-Alzheimer's disease dementia lymphocytes measured under the same lymphocytes size-measuring conditions. In another preferred embodiment, the size of the subject's lymphocytes is greater, by at least two standard deviations (and ideally by at least three standard deviations), than the size of non-Alzheimer's disease dementia lymphocytes measured under the same lymphocyte size-measuring conditions. In a further embodiment, the size of the subject's lymphocytes is greater, by at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 standard deviations, than the size of non-Alzheimer's disease dementia lymphocytes measured under the same lymphocyte size-measuring conditions.

In this method, it is envisioned that under defined lymphocyte measuring methods, a size “cutoff” can be determined whereby a lymphocyte size above that cutoff indicates that the symptomatic subject is afflicted with AD, and a lymphocyte size below that cutoff indicates that the symptomatic subject is afflicted with non-ADD.

This invention provides a method for diagnosing Alzheimer's disease in a symptomatic human subject comprising (a) (i) culturing skin fibroblasts from the subject under skin fibroblast growth-permitting conditions and (ii) measuring the growth rate of the subject's skin fibroblasts; and (b) measuring the size of the subject's skin fibroblasts under skin fibroblast size-measuring conditions, whereby the subject is afflicted with Alzheimer's disease if
the growth rate of the subject's skin fibroblasts is less, by at least one standard deviation, than the growth rate of non-Alzheimer's disease dementia skin fibroblasts cultured under the same skin fibroblast growth-permitting conditions, and/or
the size of the subject's skin fibroblasts is greater, by at least one standard deviation, than the size of non-Alzheimer's disease dementia skin fibroblasts measured under the same skin fibroblast size-measuring conditions.

The various embodiments of the growth rate- and size-based diagnostic methods above apply, mutatis mutandis, to this method.

This invention also provides a method for diagnosing Alzheimer's disease in a symptomatic human subject comprising (a) (i) culturing lymphocytes from the subject under lymphocyte growth-permitting conditions and (ii) measuring the growth rate of the subject's lymphocytes; and (b) measuring the size of the subject's lymphocytes under lymphocyte size-measuring conditions, whereby the subject is afflicted with Alzheimer's disease if
the growth rate of the subject's lymphocytes is less, by at least one standard deviation, than the growth rate of non-Alzheimer's disease dementia lymphocytes cultured under the same lymphocyte growth-permitting conditions, and/or
the size of the subject's lymphocytes is greater, by at least one standard deviation, than the size of non-Alzheimer's disease dementia lymphocytes measured under the same lymphocyte size-measuring conditions. Preferably, the lymphocytes are B lymphocytes, e.g., immortalized B lymphocytes.

The various embodiments of the growth rate- and size-based diagnostic methods above apply, mutatis mutandis, to this method.

This invention still further provides a method for diagnosing Alzheimer's disease in a symptomatic human subject comprising measuring the protein amount of the subject's skin fibroblasts under protein amount-measuring conditions, whereby the subject is afflicted with Alzheimer's disease if the protein amount of the subject's skin fibroblasts is lower, by at least one standard deviation, than the average protein amount of non-Alzheimer's disease dementia skin fibroblasts measured under the same protein amount-measuring conditions.

In this method, protein amount can be measured without first culturing the cells under growth-permitting conditions. Alternatively, protein amount can be measured after culturing the cells under growth-permitting conditions.

In a preferred embodiment, the subject is afflicted with Alzheimer's disease if the protein amount of the subject's skin fibroblasts is lower, by at least one standard deviation, than the average protein amount of non-Alzheimer's disease dementia skin fibroblasts measured under the same protein amount-measuring conditions. In another preferred embodiment, the protein amount of the subject's skin fibroblasts is lower, by at least two standard deviations (and ideally by at least three standard deviations), than the protein amount of non-Alzheimer's disease dementia skin fibroblasts measured under the same protein amount-measuring conditions. In a further embodiment, the protein amount of the subject's skin fibroblasts is lower, by at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 standard deviations, than the protein amount of non-Alzheimer's disease dementia skin fibroblasts measured under the same protein amount-measuring conditions.

This invention further provides a method for diagnosing Alzheimer's disease in a symptomatic human subject comprising measuring the protein amount of the subject's lymphocytes under protein amount-measuring conditions, whereby the subject is afflicted with Alzheimer's disease if the protein amount of the subject's lymphocytes is lower, by at least one standard deviation, than the average protein amount of non-Alzheimer's disease dementia lymphocytes measured under the same protein amount-measuring conditions. Preferably, the lymphocytes are B lymphocytes, e.g., immortalized B lymphocytes.

In this method, protein amount can be measured without first culturing the cells under growth-permitting conditions. Alternatively, protein amount can be measured after culturing the cells under growth-permitting conditions.

In a preferred embodiment, the subject is afflicted with Alzheimer's disease if the protein amount of the subject's lymphocytes is lower, by at least one standard deviation, than the average protein amount of non-Alzheimer's disease dementia lymphocytes measured under the same protein amount-measuring conditions. In another preferred embodiment, the protein amount of the subject's lymphocytes is lower, by at least two standard deviations (and ideally by at least three standard deviations), than the protein amount of non-Alzheimer's disease dementia lymphocytes measured under the same protein amount-measuring conditions. In a further embodiment, the protein amount of the subject's lymphocytes is lower, by at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 standard deviations, than the protein amount of non-Alzheimer's disease dementia lymphocytes measured under the same protein amount-measuring conditions.

This invention provides a method for diagnosing Alzheimer's disease in a symptomatic human subject comprising (a) (i) culturing skin fibroblasts from the subject under skin fibroblast growth-permitting conditions and (ii) measuring the growth rate of the subject's skin fibroblasts; (b) measuring the size of the subject's skin fibroblasts under skin fibroblast size-measuring conditions; and (c) measuring the protein amount of the subject's skin fibroblasts under protein amount-measuring conditions, whereby the subject is afflicted with Alzheimer's disease if
the growth rate of the subject's skin fibroblasts is less, by at least one standard deviation, than the growth rate of non-Alzheimer's disease dementia skin fibroblasts cultured under the same skin fibroblast growth-permitting conditions, and/or
the size of the subject's skin fibroblasts is greater, by at least one standard deviation, than the size of non-Alzheimer's disease dementia skin fibroblasts measured under the same skin fibroblast size-measuring conditions, and/or
the protein amount of the subject's skin fibroblasts is lower, by at least one standard deviation, than the average protein amount of non-Alzheimer's disease dementia skin fibroblasts measured under the same protein amount-measuring conditions.

The various embodiments of the growth rate-based, size-based and protein amount-based diagnostic methods above apply, mutatis mutandis, to this method.

This invention also provides a method for diagnosing Alzheimer's disease in a symptomatic human subject comprising (a) (i) culturing lymphocytes from the subject under lymphocyte growth-permitting conditions and (ii) measuring the growth rate of the subject's lymphocytes; (b) measuring the size of the subject's lymphocytes under lymphocyte size-measuring conditions; and (c) measuring the protein amount of the subject's lymphocytes under protein amount-measuring conditions, whereby the subject is afflicted with Alzheimer's disease if
the growth rate of the subject's lymphocytes is less, by at least one standard deviation, than the growth rate of non-Alzheimer's disease dementia lymphocytes cultured under the same lymphocyte growth-permitting conditions, and/or
the size of the subject's lymphocytes is greater, by at least one standard deviation, than the size of non-Alzheimer's disease dementia lymphocytes measured under the same lymphocyte size-measuring conditions, and/or
the protein amount of the subject's lymphocytes is lower, by at least one standard deviation, than the average protein amount of non-Alzheimer's disease dementia lymphocytes measured under the same protein amount-measuring conditions.

The various embodiments of the growth rate-based, size-based and protein amount-based diagnostic methods above apply, mutatis mutandis, to this method.

The diagnostic methods described above are also envisioned with respect to all other peripheral blood mononuclear cells (“PBMCs”), such as NK cells.

Thus, the various embodiments of the diagnostic methods above apply, mutatis mutandis, to all other PBMCs.

In addition to providing the diagnostic methods described above, this invention also provides corresponding prognostic methods, i.e., methods for determining whether an asymptomatic human subject is at risk from becoming afflicted with AD.

Specifically, this invention provides a method for determining whether an asymptomatic human subject is at risk from becoming afflicted with Alzheimer's disease comprising (a) culturing skin fibroblasts from the subject under skin fibroblast growth-permitting conditions and (b) measuring the growth rate of the subject's skin fibroblasts, whereby the subject is at risk from becoming afflicted with Alzheimer's disease if the growth rate of the subject's skin fibroblasts is greater than, yet within two standard deviations of, the growth rate of Alzheimer's disease skin fibroblasts cultured under the same skin fibroblast growth-permitting conditions.

In a preferred embodiment, the subject is at risk from becoming afflicted with Alzheimer's disease if the growth rate of the subject's skin fibroblasts is greater than, yet within 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2 or 0.1 standard deviations of, the average growth rate of Alzheimer's disease skin fibroblasts cultured under the same skin fibroblast growth-permitting conditions.

This invention also provides a method for determining whether an asymptomatic human subject is at risk from becoming afflicted with Alzheimer's disease comprising (a) culturing lymphocytes from the subject under lymphocyte growth-permitting conditions and (b) measuring the growth rate of the subject's lymphocytes, whereby the subject is at risk from becoming afflicted with Alzheimer's disease if the growth rate of the subject's lymphocytes is greater than, yet within two standard deviations of, the growth rate of Alzheimer's disease lymphocytes under the same lymphocyte growth-permitting conditions. Preferably, the lymphocytes are B lymphocytes, e.g., immortalized B lymphocytes.

In a preferred embodiment, the subject is at risk from becoming afflicted with Alzheimer's disease if the growth rate of the subject's lymphocytes is greater than, yet within 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2 or 0.1 standard deviations of, the average growth rate of Alzheimer's disease lymphocytes cultured under the same lymphocyte growth-permitting conditions.

This invention further provides a method for determining whether an asymptomatic human subject is at risk from becoming afflicted with Alzheimer's disease comprising measuring the size of the subject's skin fibroblasts under skin fibroblast size-measuring conditions, whereby the subject is at risk from becoming afflicted with Alzheimer's disease if the size of the subject's skin fibroblasts is lower than, yet within two standard deviations of, the size of Alzheimer's disease skin fibroblasts measured under the same skin fibroblast size-measuring conditions.

In a preferred embodiment, the subject is at risk from becoming afflicted with Alzheimer's disease if the size of the subject's skin fibroblasts is lower than, yet within 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2 or 0.1 standard deviations of, the average size of Alzheimer's disease skin fibroblasts measured under the same skin fibroblast size-measuring conditions.

This invention further provides a method for determining whether an asymptomatic human subject is at risk from becoming afflicted with Alzheimer's disease comprising measuring the size of the subject's lymphocytes under lymphocyte size-measuring conditions, whereby the subject is at risk from becoming afflicted with Alzheimer's disease if the size of the subject's lymphocytes is lower than, yet within two standard deviations of, the size of Alzheimer's disease lymphocytes measured under the same lymphocyte size-measuring conditions. Preferably, the lymphocytes are B lymphocytes, e.g., immortalized B lymphocytes.

In a preferred embodiment, the subject is at risk from becoming afflicted with Alzheimer's disease if the size of the subject's lymphocytes is lower than, yet within 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2 or 0.1 standard deviations of, the average size of Alzheimer's disease lymphocytes measured under the same lymphocyte size-measuring conditions.

This invention further provides a method for determining whether an asymptomatic human subject is at risk from becoming afflicted with Alzheimer's disease comprising (a) (i) culturing skin fibroblasts from the subject under skin fibroblast growth-permitting conditions and (ii) measuring the growth rate of the subject's skin fibroblasts; and (b) measuring the size of the subject's skin fibroblasts under skin fibroblast size-measuring conditions, whereby the subject is at risk from becoming afflicted with Alzheimer's disease if
the growth rate of the subject's skin fibroblasts is greater than, yet within two standard deviations of, the growth rate of Alzheimer's disease skin fibroblasts cultured under the same skin fibroblast growth-permitting conditions, and/or
the size of the subject's skin fibroblasts is lower than, yet within two standard deviations of, the size of Alzheimer's disease skin fibroblasts measured under the same skin fibroblast size-measuring conditions.

This invention further provides a method for determining whether an asymptomatic human subject is at risk from becoming afflicted with Alzheimer's disease comprising (a) (i) culturing lymphocytes from the subject under lymphocyte growth-permitting conditions and (ii) measuring the growth rate of the subject's lymphocytes; and (b) measuring the size of the subject's lymphocytes under lymphocyte size-measuring conditions, whereby the subject is at risk from becoming afflicted with Alzheimer's disease if
the growth rate of the subject's lymphocytes is greater than, yet within two standard deviations of, the growth rate of Alzheimer's disease lymphocytes cultured under the same lymphocyte growth-permitting conditions, and/or
the size of the subject's lymphocytes is lower than, yet within two standard deviations of, the size of Alzheimer's disease lymphocytes measured under the same lymphocyte size-measuring conditions. Preferably, the lymphocytes are B lymphocytes, e.g., immortalized B lymphocytes.

This invention still further provides a method for determining whether an asymptomatic human subject is at risk from becoming afflicted with Alzheimer's disease comprising measuring the protein amount of the subject's skin fibroblasts under protein amount-measuring conditions, whereby the subject is at risk from becoming afflicted with Alzheimer's disease if the protein amount of the subject's skin fibroblasts is greater than, yet within two standard deviations of, the protein amount (e.g., the average protein amount) of Alzheimer's disease skin fibroblasts measured under the same protein amount-measuring conditions.

In a preferred embodiment, the subject is at risk from becoming afflicted with Alzheimer's disease if the protein amount of the subject's skin fibroblasts is greater than, yet within 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2 or 0.1 standard deviations of, the average protein amount of Alzheimer's disease skin fibroblasts measured under the same protein amount-measuring conditions.

Finally, this invention provides a method for determining whether an asymptomatic human subject is at risk from becoming afflicted with Alzheimer's disease comprising measuring the protein amount of the subject's lymphocytes under protein amount-measuring conditions, whereby the subject is at risk from becoming afflicted with Alzheimer's disease if the protein amount of the subject's lymphocytes is greater than, yet within two standard deviations of, the protein amount (e.g., the average protein amount) of Alzheimer's disease lymphocytes measured under the same protein amount-measuring conditions. Preferably, the lymphocytes are B lymphocytes, e.g., immortalized B lymphocytes.

In a preferred embodiment, the subject is at risk from becoming afflicted with Alzheimer's disease if the protein amount of the subject's lymphocytes is greater than, yet within 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2 or 0.1 standard deviations of, the average protein amount of Alzheimer's disease lymphocytes measured under the same protein amount-measuring conditions.

The various embodiments of the growth rate-based, size-based and protein amount-based diagnostic methods above apply, mutatis mutandis, to the subject prognostic methods.

This invention will be better understood by reference to the examples which follow, but those skilled in the art will readily appreciate that the specific examples detailed are only illustrative of the invention as described more fully in the claims which follow thereafter.

### Example 1—Fibroblast and B-Lymphocyte Growth Rate Screen, Size Screen, and Total Protein Amount Screen for AD

### Introduction

We found that cell density in T25 flasks is a quantitative indicator of slow growth in AD. A manual count with a hemocytometer gives an average of over nine measurements of the #cells/ml. If one multiplies this number of cells/ml with the volume in which the cells are re-suspended after trypsnization and spinning, which is typically 3,000 ml, one can find the total number of cells in a T25 flask. If this total number of cells is then divided by 25 cm² (which is the surface area of the growth side of a T25 flask), one can find the cell density, expressed in cells/cm². The cell density in AD cell lines is typically lower than those from NDC and Non-ADD subjects.

The second observation contributing to the quantification of the slow growth in AD fibroblasts is the number of days in culture for reaching an 80-90% cell confluence. AD cell lines take a longer time to grow when compared to those from NDC and Non-ADD subjects. Cell density divided by the number of days in culture is what is defined as growth rate in FIGS. 1A-1C.

Cell size is established with the assumption that the confluence is always 85% and that this confluence is the same in each cm², out of all 25 cm² in a T25 flask. This assumption was not always correct because the cell confluence was established visually, therefore allowing a large error, i.e., 80% to 90%. A more precise method of assessing the confluence would make estimating the cell size more precise. So, 1 cm² equals (10⁴)² mm², which is 100,000,000 mm². If one takes 85% of this surface, that yields 0.85*100,000,000 (i.e., the coverage by cells in 1 cm²). Dividing the cell coverage in 1 cm² by the cell number in 1 cm² yields the average cell size depicted in FIGS. 1A-1C.

For estimating B-lymphocyte cell size, we expressed the cell density as cells/ml or cells/mm², and we assumed that we had the same volume covered with cells, i.e., 85%.

Cell size on Matrigel is measured in 10× images and then averaged via an automatic image analysis plugin in ImageJ. Typically, nine images per well are acquired in four wells at 30 minutes after plating.

### Results

The findings for the growth rate (GR) and cell size (CS) for 21 autopsy-confirmed samples (17 AD and 4 Non-ADD patients) are summarized in FIGS. 1A-1D. In FIG. 1A, a subset of just two samples is shown for four Fetal Bovine Serum (FBS) lots. It is well known that FBS, a main ingredient in the cell growth media, affects cell growth and cell size. This is demonstrated in FIG. 1A where each FBS lot is represented with a different color. However, regardless of the FBS lot, the growth rate and cell size biomarkers correctly differentiated the AD sample from the Non-ADD sample, and are separated with statistical significance (P<0.004; two-tailed equal variance).

In this study, the cut-off for the growth rate was established at 5,600, and remained the same for the subsequent study with 21 samples. The cut-off for the cell size was not established, mainly because of the imprecision in determining the cell confluence, which affects cell size determination (see above). In FIG. 1B, the growth rate and cell size for the full set of 21 samples are illustrated for just one FBS lot. The separation between AD and Non-ADD groups is statistically significant for growth rate (P<0.035 shown as a horizontal line; two tailed unequal variance) as well as for cell size (P<0.026 not shown; two tailed unequal variance). The 21 autopsy-confirmed samples show that one AD patient (black arrow) is misdiagnosed by the GR biomarker, thereby resulting in a lower sensitivity (FIG. 1D, cyan; 94%) while keeping the specificity at the maximum value (FIG. 1D; 100%) because all the Non-ADD samples show a correct diagnosis with no false positives (FIGS. 1B and 1C). Also, these 21 autopsy-confirmed samples show that one Non-ADD patient (arrow) is misdiagnosed by the CS biomarker, therefore yielding a lower specificity (FIG. 1D; 80%) while keeping the sensitivity at the maximum value (FIG. 1D; 100%) because all AD samples show a correct diagnosis with no false negatives (FIGS. 1B and 1C).

The skin fibroblast size on Matrigel at 30 minutes after plating correlates linearly with the average skin fibroblast size in T25 for the 21 autopsy-confirmed samples (FIGS. 2A and 2B).

### Combination of Growth Rate and Cell Size as a Profile Biomarker

The combination of the GR and CS biomarkers may yield a profile biomarker with 100% sensitivity and specificity. This high sensitivity and specificity can be achieved considering the two cases from FIG. 1B involving misdiagnosis using one biomarker but correct diagnosis using the other biomarker (arrows) (Chirila et al 2014), while requiring a third confirmatory biomarker, such as MI, and a simple majority rule. Alternatively, one can consider a buffer zone near the cut-off for each biomarker GR and CS, whereby each sample falling into the buffer zone would require a repeat. The buffer zone is part of the standard operating procedure for the MI assay. This zone can be easily determined when using the 6 standard deviations rule for the Gaussian distribution.

### Impaired Growth Rate in B-Lymphocytes from AD Patients

In other types of peripheral cells such as B-lymphocytes, impaired growth was observed [8] (FIGS. 3A and 3B, and 4A-4C). This impaired growth translates into an impaired growth rate (FIG. 4A) with the AD sample showing a 5- to 6-fold slower rate than the Non-ADD and NDC samples (FIG. 4B). In terms of percent change of the growth, the AD sample is 5% while the Non-ADD and NDC samples are above 25% (FIG. 4C).

The total number of cells and the number of viable cells are reduced in AD patients (triangles, FIGS. 3A and 3B) when compared with NDC (circles, FIGS. 3A and 3B) and Non-ADD patients (squares, FIGS. 3A and 3B). Impaired AD growth rate and large AD cell size were also observed in lymphocytes (FIG. 5). Thus, this effect was observed first in skin cells, and is mirrored in blood cells.

### Impaired Total Protein Amount in AD Patients

The total protein amount is low in cells from AD patients when compared with those from NDC and Non-ADD patients. This observation is true for both skin fibroblasts and B-lymphocytes, and mirrors the low cell density seen with AD patients.

However, the quantitative measure showing diagnostic separability for the 21 skin fibroblast samples with autopsy validation is the protein amount for ASPD-stimulated cells (+) (i.e., protein concentration/days in culture) (FIGS. 6A and 6B). The cut-off of 610 appears to separate all the samples, except for two marked by black arrows (indicating mixed AD dementia).

### Genetic Evidence Impaired Growth Rate and Large Cell Size in AD Patients

Fibroblast adhesion and division affect growth rate and cell size. For that reason, a subset of eight samples (6 AD and 2 Non-ADD patients) was synchronized through over-confluence and starvation, and then, the differentially expressed genes were measured. The statistically significant genes (n=21; P<0.05) were then grouped according to functional relevance for cell adhesion and cell division (Table 1). These initial findings of 21 dysregulated genes suggest the existence of a genetic basis for slow growth in AD as well as for large cell size. In synchrony with slow growth and large cell size (i.e., low cell density in AD) is low protein concentration—e.g.; low levels of PKCe. In the same subset of eight samples, several dysregulated genes (n=6; P<0.05) are related to PKC and MAPK (Table 2).

TABLE 1
Genes with functional relevance for cell adhesion and cell division

| | | | T-test |
|---|---|---|---|
| Protein | Rank | Gene Name | (2,3) |
| Pentatricopeptide Repeat Domain 2 | 1 | PTCD2 | 0.0000 |
| Coiled-Coil Domain Containing 159 | 117 | CCDC159 | 0.003 |
| Cell Division Cycle 253 | 183 | CDC25B | 0.0055 |
| TNF Superfamily Member 12 | 194 | TNFSF12 | 0.0061 |
| Glutathione 5-Transferase C-Teminal | 208 | GSTCD | 0.0066 |
| Domain Containing | | | |
| TEN1-CDK3 Readthrough (NMD | 332 | TEN1-CDK3 | 0.0115 |
| Candidate) | | | |
| Programmed Cell Death 6 | 333 | PDCD6 | 0.0117 |
| CDC42 Effector Protein 5 | 407 | CDC42EP5 | 0.0157 |
| LMCD1 Antisense RNA 1 (Head To | 469 | LMCD1-AS1 | 0.0186 |
| Head) | | | |
| CD72 Molecule | 470 | CD72 | 0.0187 |
| Cell Division Cycle 37 | 531 | CDC37 | 0.0221 |
| Cyclin Dependent Kinase 2 Associated | 550 | CDK2AP2P1 | 0.0232 |
| Protein 2 Pseudogene 1 | | | |
| Coiled-Coil Domain Containing 62 | 553 | CCDC62 | 0.0234 |
| Coiled-Coil Domain Containing 173 | 649 | CCDC173 | 0.0277 |
| Interleukin 18 Receptor 1 | 703 | IL18R1 | 0.0298 |
| Adenomatosis Poiyposis Coli Down- | 784 | APCDD1L | 0.0338 |
| Regulated 1-Like | | | |
| C2 Calcium Dependent Domain | 840 | C2CD5 | 0.0363 |
| Containing 5 | | | |
| Interleukin 17 Receptor D | 848 | IL17RD | 0.0366 |
| Coiled-Coil Domain Containing 65 | 909 | CCDC65 | 0.0393 |
| Cell Division Cycle 27 Pseudogene 2 | 978 | CDC27P2 | 0.0432 |
| Coiled-Coil Domain Containing 158 | 1006 | CCDC158 | 0.0446 |

TABLE 2
Genes with functional relevance to PKC and MAPK

| Protein | Rank | Gene Name | T-test (2,3) |
|---|---|---|---|
| Mitogen-Activated Protein Kinase 8 Interacting Protein 3 | 60 | MAPK8IP3 | 0.0014 |
| Heparin Binding EGF Like Growth Factor | 324 | HBEGF | 0.0112 |
| Heparan Sulfate Proteoglycan 2 | 369 | HSPG2 | 0.0138 |
| Solute Carrier Family 9 Member A5 | 652 | SLC9A5 | 0.0278 |
| Mitogen-Activated Protein Kinase 11 | 747 | MAPK11 | 0.0323 |
| Fos Proto-Oncogene, AP-1 Transcription Factor Subunit | 892 | FOSL1P1 | 0.0383 |

### REFERENCES

1. Chirila, F. V.; et at; “Spatiotemporal complexity of fibroblast networks screens for Alzheimer's disease”, J Alzheimer's Dis. 2013; 33(1):165-76.
2. Chirila, F. V., et al., “Fibroblast aggregation rate converges with validated peripheral biomarkers for Alzheimer's disease”, J Alzheimer's Dis. 2014; 42(4):1279-94.
3. Chen, M., et al., “Serum Starvation Induced Cell Cycle Synchronization Facilitates Human Somatic Cells Reprogramming”, PLoS ONE 7(4) (2012).
5. Baghdadchi, N., “The Effects of Serum Starvation on Cell Cycle Synchronization”, OSR Journal of Student Research, Volume 1, Article 4 (2013).
6. Hayes, O., et al., “Cell confluency is as efficient as serum starvation for inducing arrest in the G0/G1 phase of the cell cycle in granulosa and fibroblast cells of cattle”, Anim. Reprod. Sci. 87(3-4):181-92 (2005).
7. Spellman, P. T., et al, “Comprehensive identification of cell cycle-regulated genes of the yeast Saccharomyces cerevisiae by microarray hybridization”, Mol. Biol. Cell. 9(12):3273-97 (1998).
8. Chirila, F. V., et al., “B-lymphocyte imbalances of PKC□ and cellular aggregation in Alzheimer's disease patients”, Biomarkers for Alzheimer's Disease and Related Dementias, S F N 2017.

1.-6. (canceled)

7. A method for diagnosing Alzheimer's disease in a symptomatic human subject comprising measuring the protein amount of the subject's skin fibroblasts under protein amount-measuring conditions, whereby the subject is afflicted with Alzheimer's disease if the protein amount of the subject's skin fibroblasts is lower, by at least one standard deviation, than the average protein amount of non-Alzheimer's disease dementia skin fibroblasts measured under the same protein amount-measuring conditions.

8. A method for diagnosing Alzheimer's disease in a symptomatic human subject comprising measuring the protein amount of the subject's lymphocytes under protein amount-measuring conditions, whereby the subject is afflicted with Alzheimer's disease if the protein amount of the subject's lymphocytes is lower, by at least one standard deviation, than the average protein amount of non-Alzheimer's disease dementia lymphocytes measured under the same protein amount-measuring conditions.

9.-14. (canceled)

15. A method for determining whether an asymptomatic human subject is at risk from becoming afflicted with Alzheimer's disease comprising measuring the protein amount of the subject's skin fibroblasts under protein amount-measuring conditions, whereby the subject is at risk from becoming afflicted with Alzheimer's disease if the protein amount of the subject's skin fibroblasts is greater than, yet within two standard deviations of, the protein amount of Alzheimer's disease skin fibroblasts measured under the same protein amount-measuring conditions.

16. A method for determining whether an asymptomatic human subject is at risk from becoming afflicted with Alzheimer's disease comprising measuring the protein amount of the subject's lymphocytes under protein amount-measuring conditions, whereby the subject is at risk from becoming afflicted with Alzheimer's disease if the protein amount of the subject's lymphocytes is greater than, yet within two standard deviations of, the protein amount of Alzheimer's disease lymphocytes measured under the same protein amount-measuring conditions.
